(19) United States
(12) Patent Application Publication
(10) Pub. No.: US 2010/0273275 A1
(43) Pub. Date: Oct. 28, 2010

(54) Methods of determining levels of steroid fractions utilizing SHBG calculations

(75) Inventor: Bradly R. Boone, Murrieta, CA (US)

Correspondence Address: ROGER A. GILCREST, 250 WEST STREET, COLUMBUS, OH, 43215-2538, US

(73) Assignee: B&C Biotech, Murrieta, CA (US)

(21) Appl. No.: 12/803,764

(22) Filed: Jul. 6, 2010

Related U.S. Application Data
(63) Continuation of application No. 11/115,464, filed on Apr. 27, 2005, now abandoned.

(51) Int. Cl. G01N 33/53

(52) U.S. Cl. 436/501

(57) ABSTRACT

There are provided methods for assaying biological specimens for steroids (androgens and estrogens), Sex Hormone-Binding Globulin (SHBG) and Albumin in order to calculate the free and bioavailable steroid concentration based on the laws of mass action and association constants predicated on the identification that one molar equivalent of SHBG contains two molar equivalents of steroid binding sites.

### RELATED APPLICATION DATA

This application is a continuation of U.S. application Ser. No. 11/115,464, filed Apr. 27, 2005, which is hereby incorporated in its entirety herein by reference.

### FIELD OF THE INVENTION

The present invention relates generally to assay methods which allow for the detection, and quantitation of a total steroid and associated binding proteins in human biological samples, such as serum or plasma, and utilizes the laws of mass action and the binding protein association constants to calculate the concentrations of free and bioavailable steroid fractions in a sample.

### BACKGROUND OF THE INVENTION

Androgens are a group of 19-carbon (C₁₉) steroids that cause masculinization of the genital tract and the development and maintenance of male secondary sex characteristics. They also contribute to muscle bulk, bone mass, sex drive, and sexual performance in men. Testosterone is the main androgen secreted by the testes, and Dihydrotestosterone (DHT) is the main metabolite. Testosterone measurements are used to assess erectile dysfunction, infertility, gynecomastia, osteoporosis, and to monitor hormone replacement therapy. Women produce only 5% to 10% as much testosterone as do men.

Estrogens (estrone, estradiol, and estriol) are sex hormones that are responsible for the development and maintenance of the female sex organs and female secondary sex characteristics. In conjunction with progesterone, they also participate in the regulation of the menstrual cycle and breast and uterine growth, and in the maintenance of pregnancy. Estradiol is the major estrogen hormone secreted by the ovaries. Measurements of estradiol may be useful in women to assess ovarian function in women with menstrual disorders, precocious or delayed puberty, menopause, and useful in men to assess gynecomastia. Free and bioavailable estradiol levels have also shown a clinical relationship with a woman's lifetime risk of breast cancer. Measurements of estrone are primarily used for assessment of estrogen status in postmenopausal women. Normally, blood estrone levels parallel estradiol levels throughout the menstrual cycle, but at one-third to one-half their magnitude.

Greater than 97% of circulating testosterone, DHT, estradiol, and estrone are bound to plasma proteins. They are bound specifically with high affinity and low capacity to SHBG and nonspecifically with low affinity and high capacity to albumin. SHBG concentrations are increased by estrogens and therefore are higher in women than in men. They are also increased during pregnancy, oral contraceptive use, hyperthyroidism, and administration of certain antiepileptic drugs such as dilantoin. SHBG concentrations may decrease in hypothyroidism, obesity, or androgen excess.

For many years, the free fraction of steroids was thought to be the biologically active fraction. Current thought is that dissociation of albumin-bound steroid occurs within a capillary bed. Therefore the bioavailable steroid is equal to the free steroid plus the albumin-bound steroid. The albumin-bound fraction is referred to as the “non-SHBG-bound” fraction or the weakly bound fraction. In cases where the SHBG concentrations are altered, measurement of bioavailable steroid is believed to be a better reflection of steroid status.

Various methods are available for determining the concentration of free and bioavailable steroid in serum or plasma: (1) estimation of the free steroid fraction by equilibrium dialysis or ultra filtration; (2) estimate of the free steroid using a direct (“analog tracer”) immunoassay; (3) estimation of the combined free and weakly bound (“bioavailable) steroid fractions by selective precipitation of the tightly bound form; (4) calculation of androgen index using indices that reflect ratios of the steroid fractions; and (5) calculation of the free and bioavailable steroid concentrations by mathematical modeling. The mathematical modeling approach uses mass action equations to calculate free and weakly bound steroid concentrations from the total steroid, SHBG, and albumin concentrations, and from the association constants for the binding of steroids to SHBG and albumin.

All of the above methods (except 2) require that the selected total steroid concentration be measured in order to estimate the free and bioavailable steroid concentration. There are many methods for determining the total steroid concentration: (1) radioimmunoassay with extraction and/or purification, (2) mass spectrometry, and (3) immunoassays with chemiluminescence (including automated platforms). The free steroid method utilizing the direct “analog tracer” does not require the measurement of the total steroid, but comparisons with other free steroid methods has shown extremely poor correlation with the conclusion that this method not be used for measuring free steroids.

Methods using the laws of mass action modeling compared very well with equilibrium dialysis and ultra filtration methods often referred to as the “gold standards” for free steroid estimations. These “gold standard” methods are however, extremely labor intensive, time consuming, and require the use of radioactive materials which greatly reduces the availability of testing. An accurate and simple method for calculating the free and bioavailable steroid concentrations from the laws of mass action has the potential to save time, money, and reduce the usage of radioactive material.

SHBG is a protein that binds certain androgens and estrogens with high affinity and restricts the access of such steroids to tissues as long as the steroids remain bound to SHBG. In biological fluid, SHBG exists as a homodimer and each monomer comprises two laminin G-like domains. Many of the methods used to determine the SHBG concentration were calibrated based on the binding capacity of molar concentrations of androgens, like DHT, to SHBG and not based on molar concentrations of the SHBG homodimer. These methods were also extremely labor intensive and required the use of radioactive materials making them less available for testing. The prevailing assumption is that each homodimeric SHBG molecule contains a single steroid-binding site at the dimer interface. Crystallography analysis of the amino-terminal laminin G-like domain of human SHBG has shown that the dimerization and steroids-binding sites are distinct and that both monomers within the homodimeric complex are capable of binding steroid. Therefore, crystallography data demonstrates that both subunits of the SHBG homodimer bind steroid and that measurements of the molar concentration of SHBG homodimer in serum samples by binding capacity methods have been overestimated by 2-fold.

### OBJECTS OF THE INVENTION

It is an aspect of the invention to accurately estimate the free and bioavailable steroid concentrations by calculation with the laws of mass action mathematical model utilizing the total steroid, SHBG, and albumin concentrations as parameters. It is a further aspect that these parameters can be measured on automated platforms thus reducing the labor, time, money, and radioactive materials involved in the current free and bioavailable steroid methods. Ultimately, the invention and the parameters performed on an automated platform could be easily distributed to clinicians and physicians directly to reduce the testing turn-around-time to the patient, decrease labor and cost, and eliminate radioactive waste.

### SUMMARY OF THE INVENTION

Previous applications of the laws of mass action mathematical modeling assumed one steroid binding site per SHBG homodimer and used SHBG methods that were calibrated to binding capacity of androgen steroids. While these methods yielded accurate estimates of the binding capacity of SHBG for steroids required for the mathematical model, they were overestimating the SHBG concentration by 2-fold. Newer SHBG immunoassays based on detecting the concentration of the homodimer yields accurate SHBG concentrations, but the assumption of only one binding site underestimated the binding capacity and led to an overestimation of the free and bioavailable steroid concentrations using the laws of mass action mathematical model.

I have discovered a method to accurately estimate the amount of free and bioavailable steroid fractions in human serum or plasma by utilizing SHBG methods that accurately determine the homodimer concentration with the laws of mass action mathematical model assigning two steroid binding sites per SHBG homodimer. The new mathematical method can accurately estimate the free and bioavailable steroid concentrations in human samples based on the total steroid, SHBG, and albumin concentration determined by potentially simple automated methods allowing for rapid and convenient measurements.

### DETAILED DESCRIPTION OF THE INVENTION

At equilibrium, the binding of a steroid (S₁) to plasma proteins (P₁, P₂, P₃, . . . P_{N}) and the free steroid (FS₁) concentrations can be represented by:

[S₁]=[FS₁]+[P₁S₁]+[P₂S₁]+[P₃S₁]+ . . . +[P_{N}S₁] Equation 1

In serum, SHBG and albumin are the two main proteins that bind with the steroids testosterone, estradiol, estrone, and DHT, binding to transcortin or orosomucoid is negligible and can be discarded from the equation. Therefore at equilibrium the total steroid fraction concentration [S₁], SHBG-bound steroid fraction concentration [PS₁], albumin-bound steroid fraction concentration [AS₁], and the free steroid fraction concentration [FS₁] can be estimated by:

[S₁]=[FS₁]+[AS₁]+[PS₁] Equation 2

When two or more steroids and two binding proteins (SHBG and albumin) are competing for the same binding site(s) on each protein, the SHBG-bound steroid and albumin-bound steroid can be expressed by well know equations derived from the laws of mass action for multiple steroids and two binding proteins (SHBG and albumin):

[AS₁]=(N_{AS}×K_{AS1}×[A]×[FS₁])/(1+K_{AS1}×[FS₁]+K_{AS2}×[FS₂]+ . . . +K_{ASN}×[FS_{N}]) Equation 3

[PS₁]=(N_{PS}×K_{PS1}×[P]×[FS₁])/(1+K_{PS1}×[FS₁]+K_{PS2}×[FS₂]+ . . . +K_{PSN}×[FS_{N}]) Equation 4

Where, N_{AS}=number binding sites on albumin
N_{PS}=number binding sites on SHBG
K_{ASN}=association constant for albumin-binding to a steroid ligand
K_{PSN}=association constant for SHBG-binding to a steroid ligand
[A]=molar concentration of albumin
[P]=molar concentration of SHBG homodimer
[FS_{N}]=molar concentration of a free steroid ligand

Substituting Equations 3 and 4 into Equation 2 yields the mathematical model using the laws of mass action and association constants to calculate the concentration of free steroid fraction from the measured steroid, SHBG, and albumin concentrations:

[S₁]=[FS₁]+(N_{AS}×K_{AS1}×[A]×[FS₁])/(1+K_{AS1}×[FS₁]−K_{AS2}×[FS₂]+ . . . +K_{ASN}×[F_{SN}])+(N_{PS}×K_{PS1}×[P]×[FS₁])/(1+K_{PS1}×[FS₁]+K_{PS2}×[FS₂]+ . . . +K_{PSN}×[FS_{N}]) Equation 5

Noting that the number of binding sites on an SHBG homodimer is equal to 2 (N_{PS}=2), and the binding capacity of albumin can be considered infinitely high as compared to the steroid concentration such that N_{AS} can assumed to be 1 (N_{AS}=1), Equation 5 can be simplified to:

[S₁]=[FS₁]+(K_{AS1}×[A]×[FS₁])/(1+K_{AS1}×[FS₁]+K_{AS2}×[FS₂]+ . . . +K_{ASN}×[FS_{N}])(2×K_{PS1}×[P]×[FS₁])/(1+K_{PS1}×[FS₁]+K_{PS2}×[FS₂]+ . . . +K_{PSN}×[FS_{N}]) Equation 6

The bioavailable steroid fraction concentration (BS₁) can then be calculated as follows:

[BS₁]=[FS₁]+(K_{AS1}×[A]×[FS₁])/(1+K_{AS1}×[FS₁]+K_{AS2}×[FS₂]+ . . . +K_{ASN}×[FS_{N}]) Equation 7a

or

[BS₁]=[S₁]−(2×K_{PS1}×[P]×[FS₁])/+K_{PS1}×[FS₁]+K_{PS2}×[FS₂]+ . . . +K_{PSN}×[FS_{N}]) Equation 7b

The methods of the present invention can be used in all types of assays, for example but not limited to direct, competitive, simultaneous, sequential, and sandwich assays as are known in the art are within the scope of the present invention.

The most preferred biological sample type is serum; however, plasma is also acceptable where applicable to the specific method. Other biological samples can also be utilized.

Useful ranges for the association constants for binding of a steroid to SHBG are 0.5×10⁹ to 2.0×10⁹ L/M for Testosterone, 1.0×10⁹ to 3.0×10⁹ L/M for DHT, 0.2×10⁹ to 1.0×10⁹ L/M for Estradiol, and 0.05×10⁹ to 0.3×10⁹ L/M for Estrone, 0.001×10⁹ to 0.01×10⁹ L/M for Estriol, 0.01×10⁹ to 0.1×10⁹ L/M for DHEA, 0.01×10⁹ to 0.1×10⁹ L/M for Androstenedione, 0.5×10⁹ to 3.0×10⁹ L/M for Androstenediol, 0.005×10⁹ to 0.05×10⁹ L/M for Androsterone, and 0.3×10⁹ to 2.5×10⁹ L/M for Dihydroandrosterone.

Useful ranges for the association constants for binding of a steroid to albumin are 30000 to 50000 L/M for Testosterone, 50000 to 90000 L/M for DHT, 40000 to 80000 L/M for Estradiol, and 20000 to 50000 L/M for Estrone, 10000 to 30000 L/M for Estriol, 20000 to 50000 L/M for DHEA, 10000 to 30000 L/M for Androstenedione, 10000 to 30000 L/M for Androstenediol, 30000 to 50000 L/M for Androsterone, and 150000 to 200000 L/M for Dihydroandrosterone.

The steps and operations of the present invention can be performed in various orders, and the scope of the invention is not limited to the order in which the claims are written. The presently disclosed embodiments are to be considered in all respects as illustrative and not restrictive, the scope of the invention being indicated by the appended claims, rather than the foregoing description, and all changes which come within the meaning and range of equivalency of the claims are therefore intended to be embraced therein.

### Example 1

### Simple Method 1: One Steroid and Two Binding Proteins

It has been previously demonstrated that competition between other endogenous steroid hormones and androgen metabolites (S₂ to S_{N}) for binding to SHBG and albumin had very little affect on the SHBG-bound, albumin-bound, and free steroid concentrations in most clinical situations. Therefore, for practical reasons and ease of use, eliminating terms containing S₂ to S_{N} simplifies Equation 6 and allows for only the target steroid of interest to be measured in order to calculate an estimated free and bioavailable steroid levels.

Several methods can be used to determine the free steroid concentration from Equation 6 for one steroid and two binding proteins. The first method involves simplifying negligible terms and solving for [FS₁] to yielding a quadratic equation.

In Equation 3, the term (K_{AS1}×[FS₁]+K_{AS2}×[FS₂]+ . . . +K_{ASN}×[FS_{N}]) in the denominator is negligible since it is much smaller than unity due to the low affinity of albumin for the steroids. In Equation 4, the term (1+K_{PS1}×[FS₁]+K_{PS2}×[FS₂]+K_{PSN}×[FS_{N}]) in the denominator is simplified to the single steroid term (1+K_{PS1}×[FS₁]). The following equation for measuring a single steroid and the two binding proteins yields:

[S₁]=[FS₁]+(K_{AS1}×[A]×[FS₁])+(2×K_{PS1}×[P]×[FS₁])/(1+K_{PS1}×[FS₁]) Equation 8

Solving for [FS₁] in terms of a quadratic equation yields:

0=[FS₁]²×(K_{PS1}+K_{AS1}×K_{PS1}×[A])+[FS₁]×(1+K_{AS1}×[A]+2×K_{PS1}×[P]−K_{PS1}×[S₁])−[S₁] Equation 9

Using the quadratic formula to solve Equation 9, the free steroid concentration can be calculated:

[FS₁]=(−b+(b²−4×a×c)^{1/2})/(2×a)

Where, a=K_{PS1}+K_{AS1}×K_{PS1}×[A]
b=1+K_{AS1}×[A]+2×Kₚₛ₁×[P]−K_{PS1}×[S₁]
c=−[S₁]

Once the free steroid fraction concentration [FS₁] has been calculated, the bioavailable steroid fraction concentration [BS₁] can be calculated from following simplified Equation 7:

[BS₁]=[FS₁]+(K_{AS1}×[A]×[FS₁]) Equation 11a

Or

[BS₁]=[S₁]−(2×K_{PS1}×[P]×[FS₁])/(1+K_{PS1}×[FS₁]) Equation 11b

The use of the equations 10, 11a, and 11b requires that the total steroid, SHBG, and albumin quantities be expressed in terms of molar concentrations (mol/L). The resulting free steroid fraction and bioavailable steroid fraction calculated quantities are therefore derived as molar concentrations and can be converted to standardized mass concentrations by multiplying by the molecular weight of the steroid and any applicable unit conversion factor.

Table 1 illustrates a particular embodiment of the invention when the total testosterone, SHBG, and albumin concentrations are quantitated by the methods indicated, the following free testosterone and bioavailable testosterone concentrations can be calculated from the laws of mass action mathematical model Equations 10 and 11a:

### Where, K_{A}=3.6×10⁴ L/mol

Kₛ=1.0×10⁹ L/mol

TABLE 1
(Calculated Free and Bioavailable Testosterone from Simple Method 1)

| | Total | | | Calculated | Calculated |
|---|---|---|---|---|---|
| | Testos- | | | Free | Bioavailable |
| | terone | SHBG | Albumin | Testosterone | Testosterone |
| | (ng/dL) | (nmol/L) | (g/L) | (pg/mL) | (ng/dL) |
| Sample 1 | 899 | 24 | 48 | 157.5 | 410.1 |
| Sample 2 | 382 | 25 | 45 | 57.8 | 141.4 |
| Sample 3 | 640 | 42 | 40 | 71.8 | 157.0 |
| Sample 4 | 335 | 13 | 42 | 77.1 | 176.8 |
| Sample 5 | 21 | 67 | 47 | 1.3 | 3.4 |
| Sample 6 | 28 | 65 | 46 | 1.8 | 4.5 |
| Sample 7 | 47 | 82 | 41 | 2.5 | 5.7 |
| Sample 8 | 15 | 50 | 40 | 1.2 | 2.7 |
| Sample 9 | 16 | 260 | 45 | 0.3 | 0.7 |
| Sample 10 | 21 | 283 | 41 | 0.4 | 0.8 |

### Example 2

### Simple Method 2: One Steroid and Two Binding Proteins

It has been previously demonstrated that competition between other endogenous steroid hormones and androgen metabolites (S₂ to S_{N}) for binding to SHBG and albumin had very little affect on the SHBG-bound, albumin-bound, and free steroid concentrations in most clinical situations. Therefore, for practical reasons and ease of use, eliminating terms containing S₂ to S_{N} simplifies Equation 6 and allows for only the steroids of interest to be measured in order to calculate an estimated free and bioavailable steroid levels.

The second method involving Equation 6 for one steroid and two binding proteins simplifies negligible terms and solves for [FS₁] to yielding a quadratic equation.

In Equation 3, the term (1+K_{AS1}×[FS₁]+K_{AS2}×[FS₂]+ . . . +K_{ASN}×[FS_{N}]) in the denominator is simplified to the single steroid term (1+K_{AS1}×[FS₁]), and in Equation 4, the term (1+K_{PS1}×[FS₁]+K_{PS2}×[FS₂]+ . . . +K_{PSN}×[FS_{N}]) in the denominator is simplified to the single steroid term (1+K_{PS1}×[FS₁]). The following equation for measuring a single steroid and the two binding proteins yields:

[S₁]=[FS₁]+(K_{AS1}×[A]×[FS₁])/(1+K_{AS1}×[FS₁])+(2×K_{PS1}×[P]×[FS₁])/+K_{PS1}×[FS₁]) Equation 12

Arranging terms for [FS₁] yields the following equation:

0=[FS₁]³×(K_{AS1}×K_{PS1})+[FS₁]²×(K_{PS1}+K_{AS1}×K_{AS1}×K_{PS1}×[A]+2×K_{AS1}×K_{PS1}×[P]−K_{AS1}×K_{PS1}×[S₁])+[FS₁]×(1+K_{AS1}×[A]+2×K_{PS1}×[P]−K_{PS1}×[S₁]−K_{AS1}×[S₁])−[S₁] Equation 13

Noting that the term ([FS₁]³×(K_{AS1}×K_{PS1})) yields a value insignificantly small and thus can be eliminated from the equation, the quadratic formula can then be applied to solve Equation 13 for the free steroid concentration:

[FS₁]=(−b+(b²−4×a×c)^{1/2})/(2×a) Equation 14
Where, a=K_{PS1}+K_{AS1}+K_{AS1}×K_{PS1}×[A]+2×K_{AS1}×K_{PS1}×[P]−K_{AS1}×K_{PS1}×[S₁]
   b=1+K_{AS1}×[A]+2×K_{PS1}×[P]−K_{PS1}×[S₁]−K_{AS1}×[S₁]
   c=−[S₁]

Once the free steroid concentration [FS₁] has been calculated, the bioavailable steroid concentration [BS₁] can be calculated from following simplified Equation 7:

[BS₁]=[FS₁]+(K_{AS1}×[A]×[FS₁])/(1+K_{AS1}×[FS₁]) Equation 15a

or

[BS₁]=[S₁]−(2×K_{PS1}×[P]×[FS₁])/(1+K_{PS1}×[FS₁]) Equation 15b

The use of the equations 14, 15a, and 15b requires that the total steroid, SHBG, and albumin quantities be expressed in terms of molar concentrations (mol/L). The resulting free and bioavailable steroid calculated quantities are therefore derived as molar concentrations and can be converted to standardized mass concentrations by multiplying by the molecular weight of the steroid and any applicable unit conversion factor.

Table 2 illustrates a particular embodiment of the invention where the total testosterone, SHBG, and albumin concentrations are quantitated by the methods indicated, the following free testosterone and bioavailable testosterone concentrations can be calculated from the laws of mass action mathematical model Equations 14 and 15a:

### Where, K_{A}=3.6×10⁴ L/mol

K_{S}=1.0×10⁹ L/Mol

TABLE 2
(Calculated Free and Bioavailable Testosterone from Simple Method 2)

| | Total | | | Calculated | Calculated |
|---|---|---|---|---|---|
| | Testos- | | | Free | Bioavailable |
| | terone | SHBG | Albumin | Testosterone | Testosterone |
| | (ng/dL) | (nmol/L) | (g/L) | (pg/mL) | (ng/dL) |
| Sample 1 | 899 | 24 | 48 | 157.5 | 410.1 |
| Sample 2 | 382 | 25 | 45 | 57.8 | 141.4 |
| Sample 3 | 640 | 42 | 40 | 71.8 | 157.0 |
| Sample 4 | 335 | 13 | 42 | 77.1 | 176.8 |
| Sample 5 | 21 | 67 | 47 | 1.3 | 3.4 |
| Sample 6 | 28 | 65 | 46 | 1.8 | 4.5 |
| Sample 7 | 47 | 82 | 41 | 2.5 | 5.7 |
| Sample 8 | 15 | 50 | 40 | 1.2 | 2.7 |
| Sample 9 | 16 | 260 | 45 | 0.3 | 0.7 |
| Sample 10 | 21 | 283 | 41 | 0.4 | 0.8 |

Note that Simple Method 1 and 2 yield identical free and bioavailable testosterone results.

### Example 3

### Multiple Steroid Method: Multiple Steroids and Two Binding Proteins

In certain clinical situations where potential interference is cause by massive levels of steroids binding to SHBG, it may be prudent to account for the interfering steroids when determining a free and bioavailable steroid concentrations. Using Equation 6 and noting as in Example 1 that the term (K_{AS1}×[FS₁]+K_{AS2}×[FS₂]+ . . . +K_{ASN}×[FS_{N}]) in the denominator is negligible since it is much smaller than unity due to the low affinity of albumin for steroids, the following mass action equation can be formulated to calculate the free steroid concentration of S₁ while compensating for the interference of steroids S₂ to S_{N}:

[S₁]=[FS₁]+(K_{AS1}×[A]×[FS₁])+(2×K_{PS1}×[P]×[FS₁])/(1+K_{PS1}×[FS₁]−K_{PS2}×[FS₂]+ . . . +K_{PSN}×[FS_{N}]) Equation 16

For ease of use, the terms (K_{PS2}×[FS₂]+ . . . +K_{PSn}×[FSₙ]) can be written as Σ(K_{PS2-N}×[FS_{2-N}]).

Solving for [FS₁] yields the following quadratic equation:

0=[FS₁]²×(K_{PS1}+K_{AS1}×K_{PS1}×[A])+[FS₁]×(1+K_{AS1}×[A]+2×K_{PS1}×[P]+Σ(K_{PS2-N}×[FS_{2-N}])+K_{AS1}×[A]×Σ(K_{PS2-N}×[FS_{2-N}])−K_{PS1}×[S₁])−[S₁]−[S₁]×Σ(K_{PS2-N}×[FS_{2-N}]) Equation 17

Using the quadratic formula to solve Equation 10, the free steroid concentration can be calculated:

[FS₁]=(−b+(b²−4×a×c)^{1/2})/(2×a) Equation 18

Where, a=K_{PS1}×K_{PS1}×[A]
b=1+K_{AS1}×[A]+2×K_{PS1}×[P]+Σ(K_{PS2-N}×[FS_{2-N}])+K_{AS1}×[A]×Σ(K_{PS2-N}×[FS_{2-N}])−K_{PS1}×[S₁])
c=−[S₁]−[S₁]×Σ(K_{PS2-N}×[FS_{2-N}])

In order to solve Equation 18, the free steroid concentrations of steroids S₂ to S_{N} need to be determined. By measuring the total steroid concentration for S₂ to S_{N} along with the SHBG and albumin concentrations, the simple method from Example 1 (Equation 10) can be used to estimate the free steroid concentrations. Once the free steroid concentration are determined, they can be use calculate the term Σ(K_{PS2-N}×[FS_{2-N}] and thus calculate the free steroid concentration of S₁.

Once the free steroid concentration [FS₁] has been calculated, the bioavailable steroid concentration [BS₁] can be calculated from Equation 11a.

The use of the equations 18 and 11a requires that the total steroids (S₁ to S_{N}), free steroid (FS₁ to FS_{N}), SHBG, and albumin quantities be expressed in terms of molar concentrations (mol/L). The resulting free (FS₁) and bioavailable (BS₁) steroid calculated quantities are therefore derived as molar concentrations and can be converted to standardized mass concentrations by multiplying by the molecular weight of the steroid and any applicable unit conversion factor.

Tables 3 through 6 illustrate a particular embodiment of the invention for determining the free testosterone concentration from the total testosterone, SHBG, and albumin concentrations while compensating for 7 competing steroid analytes (DHT, Estrone, Estradiol, Dehydroepiandrosterone (DHEA), Androstenedione (A-dione), Dihydroandrosterone (DiH-A-one), and Androstenediol (A-diol).

TABLE 3
(Measured Total Testosterone, SHBG, Albumin, and Total Steroid Analytes)

| | | | | | | | | | Total | |
|---|---|---|---|---|---|---|---|---|---|---|
| | Total | | | Total | Total | Total | Total | Total | DiH-A- | Total |
| | Testo | SHBG | Alb | DHT | Estrone | Estradiol | DHEA | A-dione | one | A-diol |
| | (ng/dL) | (nmol/L) | (g/L) | (pg/mL) | (pg/mL) | (pg/mL) | (pg/mL) | (pg/mL) | (pg/mL) | (pg/mL) |
| Sample 1 | 899 | 24 | 48 | 621 | 52 | 42 | 8600 | 2120 | 450 | 1550 |
| Sample 2 | 382 | 25 | 45 | 504 | 21 | 16 | 3200 | 1390 | 510 | 1100 |
| Sample 3 | 640 | 42 | 40 | 412 | 44 | 25 | 5810 | 1860 | 320 | 1210 |
| Sample 4 | 335 | 13 | 42 | 322 | 24 | 31 | 4400 | 950 | 650 | 850 |
| Sample 5 | 21 | 67 | 47 | 95 | 66 | 120 | 3420 | 1310 | 420 | 660 |
| Sample 6 | 28 | 65 | 46 | 186 | 108 | 86 | 2920 | 1620 | 490 | 820 |
| Sample 7 | 47 | 82 | 41 | 210 | 166 | 304 | 4120 | 1250 | 320 | 390 |
| Sample 8 | 15 | 50 | 40 | 139 | 84 | 216 | 3930 | 860 | 310 | 440 |
| Sample 9 | 16 | 260 | 45 | 320 | 6600 | 12000 | 4950 | 5980 | 360 | 220 |
| Sample 10 | 21 | 283 | 41 | 415 | 7800 | 14000 | 5510 | 6350 | 390 | 190 |

TABLE 4
(Calculated Free Steroid Analyte Concentrations from Simple Method 1)

| | Free | Free | Free | Free | Free | Free | Free |
|---|---|---|---|---|---|---|---|
| | DHT | Estrone | Estradiol | DHEA | A-dione | DiH-A-one | A-diol |
| | (pg/mL) | (pg/mL) | (pg/mL) | (pg/mL) | (pg/mL) | (pg/mL) | (pg/mL) |
| Sample 1 | 4.1 | 1.4 | 0.6 | 270.4 | 130.2 | 2.4 | 19.3 |
| Sample 2 | 3.3 | 0.6 | 0.2 | 105.6 | 89.8 | 2.8 | 13.0 |
| Sample 3 | 1.9 | 1.2 | 0.3 | 197.0 | 124.0 | 1.5 | 9.1 |
| Sample 4 | 3.2 | 0.8 | 0.6 | 163.0 | 68.2 | 4.5 | 17.4 |
| Sample 5 | 0.3 | 1.4 | 1.1 | 92.7 | 70.9 | 1.4 | 3.1 |
| Sample 6 | 0.6 | 2.3 | 0.8 | 80.9 | 89.7 | 1.7 | 4.0 |
| Sample 7 | 0.6 | 3.4 | 2.6 | 116.7 | 71.0 | 1.0 | 1.5 |
| Sample 8 | 0.6 | 2.1 | 2.5 | 128.4 | 55.6 | 1.3 | 2.7 |
| Sample 9 | 0.3 | 64.5 | 42.7 | 81.4 | 207.5 | 0.5 | 0.3 |
| Sample 10 | 0.4 | 73.3 | 47.1 | 89.8 | 219.4 | 0.5 | 0.2 |

TABLE 5
(Calculated Free Testosterone from Multiple Steroid Method compared with Simple
Method 1)

| | | | | | Multiple | |
|---|---|---|---|---|---|---|
| | | | | Simple | Steroid | |
| | Total | | | Method Free | Method Free | Difference |
| | Testosterone | SHBG | Albumin | Testosterone | Testosterone | Between |
| | (ng/dL) | (nmol/L) | (g/L) | (pg/mL) | (pg/mL) | Methods |
| Sample 1 | 899 | 24 | 48 | 157.47 | 170.97 | 8.6% |
| Sample 2 | 382 | 25 | 45 | 57.76 | 62.17 | 7.6% |
| Sample 3 | 640 | 42 | 40 | 71.81 | 78.03 | 8.7% |
| Sample 4 | 335 | 13 | 42 | 77.14 | 82.34 | 6.7% |
| Sample 5 | 21 | 67 | 47 | 1.32 | 1.38 | 4.6% |
| Sample 6 | 28 | 65 | 46 | 1.82 | 1.91 | 5.2% |
| Sample 7 | 47 | 82 | 41 | 2.54 | 2.67 | 4.9% |
| Sample 8 | 15 | 50 | 40 | 1.24 | 1.30 | 5.2% |
| Sample 9 | 16 | 260 | 45 | 0.29 | 0.34 | 15.1% |
| Sample 10 | 21 | 283 | 41 | 0.36 | 0.42 | 16.7% |

TABLE 6
(Steroid Association Constants and Molar
Conversions used for Calculations)

| | K_{PS} | K_{AS} | Molar Conversion |
|---|---|---|---|
| Steroid | [M⁻¹] × 10⁹ | [M⁻¹] | (pg/mL/mol/L) × 10¹¹ |
| Testosterone | 1.0 | 36000 | 2.884 |
| Dihydrotestosterone | 2.0 | 79800 | 2.904 |
| (DHT) | | | |
| Estrone | 0.15 | 40000 | 2.704 |
| Estradiol | 0.5 | 60000 | 2.724 |
| Dehydroepiandrosterone | 0.066 | 40000 | 2.884 |
| (DHEA) | | | |
| Androstenedione | 0.029 | 20000 | 2.864 |
| (A-dione) | | | |
| Dihydroandrosterone | 1.3 | 180000 | 2.924 |
| (DiH-A-one) | | | |
| Androstenediol | 1.5 | 20000 | 2.904 |
| (A-diol) | | | |

Dunn et al. (1981)
Moll et al. (1981)
Rosner (1991)

### Example 4

### Calculation of Free and Bioavailable Testosterone Using Fixed Albumin Concentration

It has been previously demonstrated that the fluctuation in the albumin concentration in normal humans has very little affect on the SHBG-bound, albumin-bound, and free steroid concentrations in most clinical situations. Therefore, pre-selecting a fixed concentration for the albumin measurement in the laws of mass action mathematical model allows for one less analyte to be measured without significant impact on the free and bioavailable steroid concentrations.

Table 7 illustrates a particular embodiment of the invention when the total testosterone and SHBG concentrations are quantitated by the methods indicated and the albumin concentration is replaced with a pre-selected fixed amount, the following free testosterone and bioavailable testosterone concentrations can be calculated from the laws of mass action mathematical model Equation 10 and 11a (Simple Method 1):

### Where, K_{A}=3.6×10⁴ L/mol

K_{S}=1.0×10⁹ L/mol
Albumin=43 g/L

TABLE 7
(Calculated Free and Bioavailable Testosterone from
Simple Method 1 with fixed Albumin Concentration)

| | | | Calculated | Calculated |
|---|---|---|---|---|
| | Total | | Free | Bioavailable |
| | Testosterone | SHBG | Testosterone | Testosterone |
| | (ng/dL) | (nmol/L) | (pg/mL) | (ng/dL) |
| Sample 1 | 899 | 24 | 167.0 | 391.4 |
| Sample 2 | 382 | 25 | 58.8 | 137.8 |
| Sample 3 | 640 | 42 | 70.4 | 164.9 |
| Sample 4 | 335 | 13 | 76.1 | 178.4 |
| Sample 5 | 21 | 67 | 1.3 | 3.1 |
| Sample 6 | 28 | 65 | 1.8 | 4.3 |
| Sample 7 | 47 | 82 | 2.5 | 5.9 |
| Sample 8 | 15 | 50 | 1.2 | 2.9 |
| Sample 9 | 16 | 260 | 0.3 | 0.7 |
| Sample 10 | 21 | 283 | 0.4 | 0.8 |

What is claimed is:

1. A method of determining a free concentration of testosterone and a bioavailable concentration of testosterone within a biological sample, the method comprising:
(a) using an analytical method to measure (i) a total concentration of testosterone, (ii) a concentration of Sex Hormone Binding Globulin (SHBG), and (iii) a concentration of albumin, all within the biological sample; and
(b) calculating said free concentration and said bioavailable concentration of testosterone in said biological sample with an equilibrium reaction equation using the measured total concentration of testosterone, the measured concentration of SHBG, wherein said equilibrium reaction equation contains a term that mathematically attributes two steroid binding sites per molecule of said SHBG within said equilibrium reaction equation, and the measured concentration of albumin, with at least one association constant for binding of testosterone to SHBG, and at least one association constant for binding of testosterone to albumin, wherein said free concentration of testosterone is obtained using an equation of the form:
[S]=[FS]+(K_{AS}×[A]×[FS])+(2×K_{PS}×[P]×[FS])/(1+K_{PS}×[FS])
and wherein said bioavailable concentration of testosterone is obtained using an equation of the form:
[BS]=[FS]+(K_{AS}×[A]×[FS])
wherein
K_{AS}=association constant for albumin-binding to testosterone
K_{PS}=association constant for SHBG-binding to testosterone
[A]=molar concentration of albumin
[P]=molar concentration of SHBG homodimer
[FS]=molar concentration of a free testosterone
[S]=molar concentration of a testosterone
[BS]=molar concentration of a bioavailable testosterone.

2. The method of claim 1 wherein said analytical method for measuring said SHBG concentration is immunoassay and measures a homodimer SHBG concentration.

3. The method of claim 1 wherein said biological sample is a human biological fluid selected from one of serum or plasma.

4. The method of claim 1 wherein said at least one association constant for binding of testosterone to said SHBG is within a range of 0.5.×10⁹ to 2.0×10⁹ L/M for Testosterone.

5. The method of claim 1 wherein said at least one association constant for binding of testosterone to said albumin is within a range of 30000 to 50000 L/M for Testosterone.
